(19) United States
(12) Patent Application Publication
(10) Pub. No.: US 2026/0166218 A1
(43) Pub. Date: Jun. 18, 2026

(54) PUMPING SEGMENT RECOVERY MECHANISM

(71) Applicant: CareFusion 303, Inc., San Diego, CA (US)

(72) Inventor: Chun Keat OOI, Limerick (IE)

(21) Appl. No.: 19/125,034

(22) PCT Filed: Oct. 28, 2022
(86) PCT No.: PCT/US2022/048282

§ 371 Date: Apr. 28, 2025

(51) Int. Cl. A61M 5/142

(52) U.S. Cl. CPC A61M 5/14228

(57) ABSTRACT

A pumping segment recovery device coupled to a pumping mechanism is disclosed. The pumping segment recovery device includes a first arm segment, a second arm segment, a tube receiving space between the first arm and the second arm, and a connecting segment coupling the first arm segment with the second arm segment. The connecting segment is arranged below the tube receiving space. The first arm segment includes a first flared end to contact a pumping element before the pumping element contacts a fluid tube received in the tube receiving space, and the second arm segment includes a second flared end to contact the pumping element before the pumping element contacts the fluid tube received in the tube receiving space.

### TECHNICAL FIELD

The present disclosure generally relates to an intravenous (IV) set or infusion pump flow control, and in particular a pumping segment recovery device coupled along a fluid path of an infusion pump that is configured to rebound an IV tube.

### BACKGROUND

Peristaltic pumping mechanisms in Large-Volume Pumps (LVPs) generally include one or more pump fingers which compress on a pumping segment of IV set tubing (which is generally made of elastomeric polymer like silicone rubber or PVC). When fully compressed, the tubing is generally flattened (e.g., inner surface of the tube is flattened against itself) to not allow any fluid through. When the pump fingers lifts, the pumping segment is generally expected to recover or bounce back quickly or immediately to its original round cross-section purely by its elastic material property. In conventional pumping mechanisms, this does not happen quick enough due to stiction on inner tube surfaces (which can be worsened by multiple factors, such as high-friction elastomeric material, PVC plasticizer migration, large number of cycles, tube aging, long compression time, high compression force, short cycle time during high flow rates, partial vacuum pressure in upstream tube, etc.). When the pumping segment does not recover immediately, there is under-infusion as less fluid is delivered to a patient than is expected or calculated by pumping software (which assumes quick/immediate tube elastic recovery).

Thus, it is desirable to provide a pump device that improves the reliability of flow rates and volumes.

### SUMMARY

Various implementations of systems, methods, and devices within the scope of the appended claims each have several aspects, no single one of which is solely responsible for the attributes described herein. Without limiting the scope of the appended claims, after considering this disclosure, and particularly after considering the section entitled “Detailed Description” one will understand how the aspects of some implementations are used to provide accurate and consistent flow rates. One or more implementations provide a pumping segment recovery device coupled along a fluid path of pump device that includes one or more pumping elements. The pumping segment recovery device is configured to receive an IV tube within an tube receiving portion. The pumping segment recovery device includes at least two arm segments that do not contact the IV tube when the IV tube is in an uncompressed state. When a pumping element of the pumping device is engaged and moves toward the IV tube to compress the IV tube, the pumping element makes contact with the pumping segment recovery device separating at least two arm segments of the pumping segment recovery device before compressing the IV tube. When the pumping element is withdrawn (e.g., moved away from the IV tube allowing the IV tube to return to its uncompressed state), the least two arm segments of the pumping segment recovery device rebound (or return to their initial positions) and apply forces to opposing sides of the IV tube, which causes the IV tube to return to its uncompressed state quickly.

In some implementations, the pumping segment recovery device is a generally-symmetrical elastic or resilient member mounted close to a pumping mechanism or pump finger. The pumping segment recovery device includes angled surfaces or flared arms (e.g., two opposing s-shaped members). In some implementations, the pumping segment recovery device is a leaf spring, compression spring, or other similar device. In some implementations, the pump mechanism or pump finger has generally-symmetrical chamfered edges. In a neutral position, the pumping segment recovery device does not contact a round outer diameter of tubing, or the pump finger. When the pump finger is moved down onto the tubing (compressing the tubing), the edges (e.g., chamfered edges) of the pump finger push open the angled surfaces of pumping segment recovery device at the same time as the pump finger bottom surface compresses on the tubing. While the pump finger compresses the tubing, the pumping segment recovery device does not contact the outer diameter of the tubing. When pump finger moves up away from tubing, the pumping segment recovery device begins to close back to its original shape and pushes the outer diameter of the flattened tubing inwards to overcome any possible stiction on tube inner surfaces. Once tube internal stiction of the flattened tubing is released, the elastic tubing material will naturally recover to its original round cross-section. The pumping segment recovery device provides reliable and quick recovery of a pumping segment every cycle, minimizing under-infusion due to this stiction issue.

The pumping segment recovery device can be formed of metal, such as stainless steel, or any materials with elastic recovery properties, creep resistance, and fatigue resistance that maintain robust and consistent material behavior for the same number of cycles, stress, aging, etc. as the tubing material. This further ensure reliable and quick recovery of pumping segment from any potential internal surface stiction.

The foregoing and other features, aspects and advantages of the disclosed implementations will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate implementations of the disclosure and together with the description serve to explain the principles of the disclosure.

FIG. 1 shows a patient care system, in accordance with various aspects of the present disclosure.

FIG. 2 show operation of a conventional pumping device.

FIG. 3 illustrates pumping segment recovery mechanisms, in accordance with some implementations.

FIG. 4 illustrates a pumping segment recovery device incorporated in an infusion deice, in accordance with some implementations.

FIG. 5 illustrates the pumping segment recovery device incorporated in another pumping device, in accordance with some implementations.

FIG. 6 illustrates a flow diagram of a method for operating a pumping device including a pumping segment recovery device, in accordance with some implementations.

FIG. 7 is a conceptual diagram illustrating an example electronic system for controlling a flow rate of a medical device, according to aspects of the subject technology.

Like reference numerals refer to corresponding parts throughout the drawings.

### DETAILED DESCRIPTION

A pumping segment recovery mechanism is disclosed. The pumping segment recovery mechanism is incorporated into an infusion pump, for example, within one or more pumping mechanisms, and is configured to facilitate the return of an IV set to its original (uncompressed) state after it has been compressed. During operation, the pumping segment recovery mechanism is configured to releasably couple to the one or more pump elements without constricting the flow of the IV set and/or interfering with operation of the one or more pump elements. The pumping segment recovery mechanisms reduce or prevent the IV set from being compressed before it returns to its original state thereby improving the accuracy and consistency of the flow rate generated by a pump device.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to controlling and maintaining a flow rate during the administration of medical fluid using the disclosed pumping segment recovery mechanism incorporated in pump device, it is to be understood that this description is only an example of usage and does not limit the scope of the claims. Various aspects of the disclosed pumping segment recovery mechanism may be used in any application where it is desirable to control a fluid flow rate.

The disclosed pumping segment recovery mechanisms overcome several challenges discovered with respect to certain conventional pump devices. One challenge with certain conventional pump devices is that the accuracy and consistency of the flow rate can change with time and/or based on the selected flow rate. Because inaccuracies and/or inconsistencies in pump devices can alter the flow rate of the administered medical fluid, pumps of the pump devices need to be monitored to ensure the efficacy of a treatment provided by the pump devices. Monitoring the performance of the pumps of the pump devices can be expensive and challenging requiring additional sensors and/or programs to constantly track performance of the pumps. Therefore, in accordance with the present disclosure, it is advantageous to provide pumping segment recovery mechanisms as described herein that can be included in pump devices to substantially reduces or eliminate inaccuracies and inconsistencies in the generated flow rates of pump devices. The disclosed pumping segment recovery mechanisms can be coupled to different portions of pump elements of a pump device and provide additional forces assist the IV set in rebounding (to a resting state) before it is compressed thereby improving the accuracy and consistency of the generated flow rate.

An example of a pump device that improves the accuracy and consistency of the generated flow rates is now described.

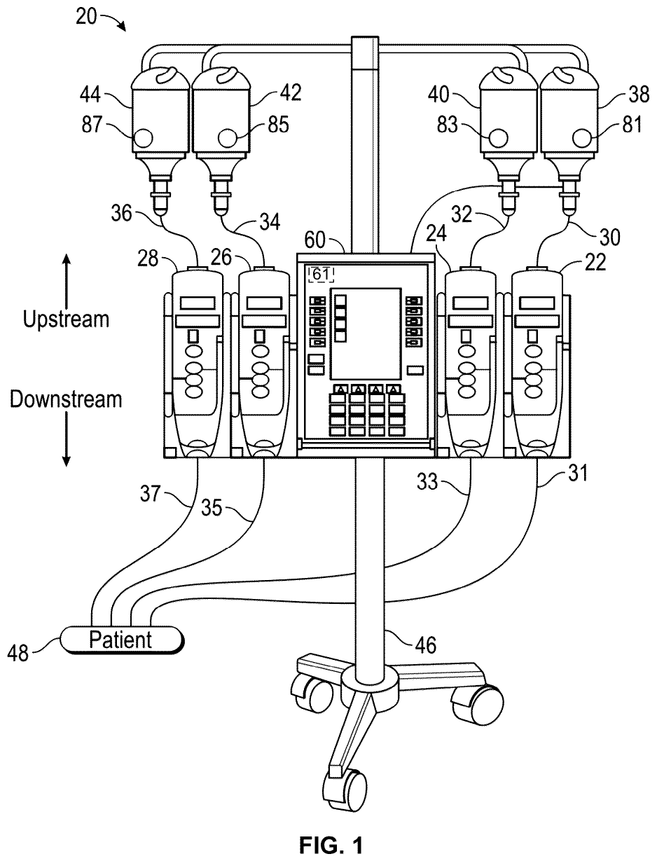

FIG. 1 shows a patient care system, in accordance with various aspects of the present disclosure. The patient care system 20 includes four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump devices can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, tabletop, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a controller 60. In some implementations, the controller 60 is communicatively coupled to memory 61 storing one or more instructions for operating the infusion pumps 22, 24, 26, and 28 and/or other collected data as described below.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typically, medical fluid administration sets have more parts than are shown in FIG. 1. Many have check valves, drip chambers, valves with injection ports, connectors, and other devices well known to those skilled in the art. These other devices have not been included in the drawings so as to preserve clarity of illustration.

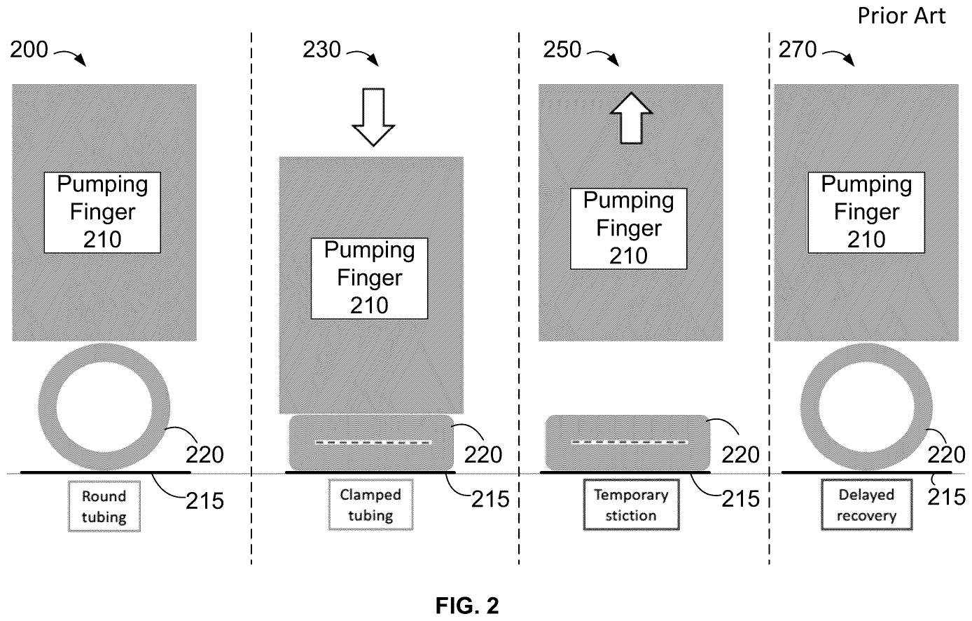

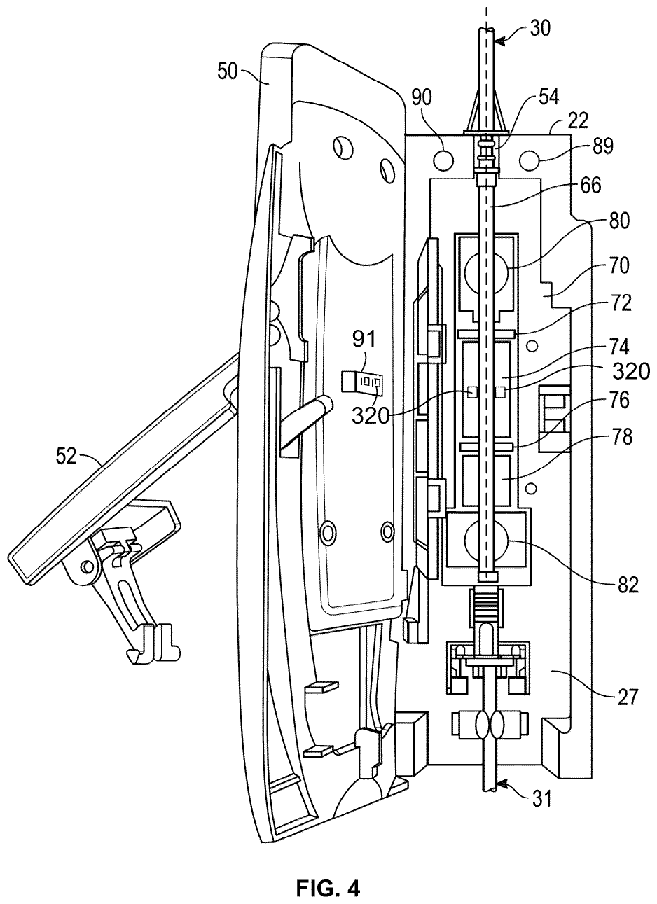

FIG. 2 show operation of a conventional pumping device. A first sequence 200 shows a pumping finger 210 and a pumping surface 215 (e.g., platen) of the conventional pumping device and an IV tube 220. The pumping finger 210 and pumping surface 215 are configured to compress the IV tube 220 and cause fluid to flow through the IV tube 220. In particular, the pumping finger 210 is an actuated component of the conventional pumping device, such as a pumping finger or similar device, and the pumping surface 215 is a stationary surface of the conventional pumping device that operatively compress the IV tube 220. As will be described further with respect to FIG. 4, the pumping surface may be integrated with a door of the pump.

As shown in second sequence 230, the pumping finger 210 is actuated (e.g., moved toward the pumping surface 215) to compresses the IV tube 220 against the pumping surface 215. In the third sequence 250, after the pumping finger 210 is actuated and returned to its starting position (e.g., moved away from the pumping surface 215), the IV tube 220 begins to return to its original (uncompressed) state. However, in many instances, the IV tube 220 is not able to return to its original state before the pumping finger 210 is actuated a subsequent time and compresses the IV tube 220 a second time. As shown in the third sequence 250, the IV tube 220 can experience stiction which further impedes the IV tube 220's ability to return to its original state before the pumping finger 210 is actuated a subsequent time. Specifically, as shown in a fourth sequence 270, IV tube 220 has a delayed recovery (i.e., the IV tube 220 does not immediately return to its original state). Because the IV tube 220 is not able to return to its original state before the pumping finger 210 compresses the IV tube 220 a second time, the flow rate generated by the conventional pumping device can be inaccurate. In particular, the conventional pumping device relies on the IV tube 220 being able to rebound quickly enough before it is compressed again to provide consistent flow rates, which is not always possible. As faster flow rates are targeted by the medical device, less times is allotted for the IV tube 220 to return to its original state before it is compressed a second time. Further, due to variations in tube diameter, wall thickness, and a durometer along with additional mechanical properties; the displaced fluid volume per stroke varies in conventional pumping device solutions.

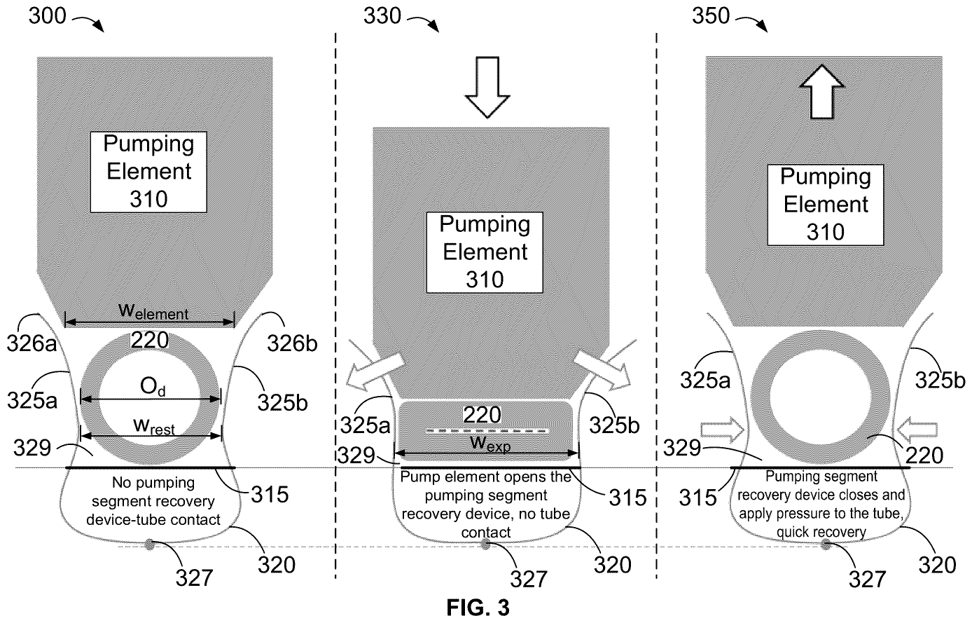

FIG. 3 illustrates pumping segment recovery mechanisms, in accordance with some implementations. A first example sequence 300 shows the disclosed pumping segment recovery mechanism, including a pumping segment recovery device 320 incorporated in a pumping device (e.g., infusion pumps 22, 24, 26, and 28; FIG. 1). In some implementations, the pumping device includes a first pumping element 310 and a second pumping element 315 configured to compress an IV tube 220. When the pumping element 310 is actuated and the tube 220 is compressed, fluid is caused to flow through the IV tube 220. In some implementations, the first pumping element 310 is a pump finger including chamfered ends (adjacent to the second pumping element 315), square ends, circular ends. Alternatively, in some implementations, the first pumping element 310 is a wedge, a cone, a ball, or other shape. Similarly, in some implementations, the second pumping element 315 is a stationary surface of the of the pumping device. In some implementations, the first pumping element 310 is actuated to cause the compression of the tube 220. In some implementations, the second pumping element 315 is actuated to cause the compression (e.g., by moving the surface against the element 310). In some implementations, both the first pumping element 310 and the second pumping element 315 are actuating components. Alternatively, as shown in FIG. 3, at least one pumping element is stationary (e.g., the second pumping element 315 is a flat planar surface).

In some implementations, the pumping segment recovery device 320 is a substantially symmetrical. In some implementations, the pumping segment recovery device 320 is formed of metal (e.g., stainless steel), elastomers, or any other material with a predetermined elasticity to return to its original shape as discussed in detail below. The material properties of the pumping segment recovery device 320 are such that the recovery properties, creep resistance, and fatigue resistance of the pumping segment recovery device 320 maintain a robust and consistent material behavior for the same number of cycles, stress, aging, etc. as the pumping segment. In some implementations, the pumping segment recovery device 320 is a leaf spring, compression springs, extension springs, etc.

In the depicted example, the pumping segment recovery device 320 includes a first arm segment 325a, a second arm segment 325b, and a tube receiving space 329 between the first arm and the second arm segments 325a and 325b. The pumping segment recovery device 320 is configured to receive and hold an IV tube 220 within the tube receiving space 329. The connecting segment 327 couples the first arm segment 325a with the second arm segment 325b. Further, the connecting segment 327 is arranged below the tube receiving space 329. The first arm segment 325a includes a first flared end 326a that is configured contact a pumping element (e.g., first pumping element 310) before the pumping element contacts a fluid tube (e.g., IV tube 220) received in the tube receiving space 329, and the second arm segment 325b includes a second flared end 326b configured to contact the pumping element before the pumping element contacts the fluid tube received in the tube receiving space 239. In some implementations, the first arm segment 326a has a first s-shape and the second arm segment 326b has a second s-shape opposite the first s-shape. In some implementations, the pumping segment recovery device 320 can include cams to interact with the different pumping elements.

As shown in the first sequence 200, the first arm segment 325a and the second arm segment 325b of the pumping segment recovery device 320 are separated by a gap width (e.g., wᵣₑₛₜ) greater than an outer diameter (O_{d}) of the IV tube 220 received in the tube receiving space 329. In this way, the pumping segment recovery device 320 does not make contact with the IV tube 220 before the IV tube 220 is compressed. To this point, the gap width (e.g., wᵣₑₛₜ) of the pumping segment recovery device 320 can be predetermined base on the type and the size of IV tube 220 used to facilitate flow of medical fluid to a patient 48 (FIG. 1).

In some implementations, the first and second flared ends 326a and 326b are separated by a width equal to or greater than a width of a pumping segment. For example, in the FIG. 3, the separation width of the first and second flared ends 326a and 326b is equal to or greater than a width (e.g., wₑₗₑₘₑₙₜ) of the first pumping element 310. In some implementations, the separation width of the first and second flared ends 326a and 326b is greater than the gap width (wᵣₑₛₜ) of the of the pumping segment recovery device 320 at rest. In some implementations, the gap width (wᵣₑₛₜ) of the of the pumping segment recovery device 320 is predefined based on the respective s-shapes of the first and second arm segments 325a and 325b. Alternatively or additionally, in some implementations, the gap width (wᵣₑₛₜ) of the of the pumping segment recovery device 320 is predefined based on the connecting segment 327.

Turning to a second sequence 330, the first pumping element 310 is actuated toward the second pumping element 315 thereby compressing the IV tube 220 against the second pumping element 315. When the first pumping element 310 engages and is actuated towards the second pumping element 315 (e.g., contacting the IV tube 220 received in the tube receiving space 329), the first arm segment 325a and the second arm segment 325b separate to at least the width of a portion of the first pumping element 325a (e.g. wₑₓₚ). The first pumping element 310 contacts the first and second flared ends 326a and 326b when first contacting the pumping segment recovery device 320 and glide along the first arm segment 325a and the second arm segment 325b while traveling toward the second pumping element 315 and before contacting the IV tube 220. The force required to separate the first arm segment 325a and the second arm segment 325b is substantially less than the force that is used to actuate the pumping element. More specifically, the first arm segment 325a and the second arm segment 325b do not substantially impede actuation of a pumping element and do not reduce the force applied to the IV tube 220 and/or change a targeted flow rate. In some implementations, the force applied by the pumping element can be adjusted to compensate for the resistance provided by the first arm segment 325a and the second arm segment 325b.

When the first arm segment 325a and the second arm segment 325b are separated to at least the width of a portion of the first pumping element 325a (e.g. wₑₓₚ), the first arm segment 325a and the second arm segment 325b do not contact the IV tube 220. This further ensures that the first arm segment 325a and the second arm segment 325b do not interfere with the flow generated by compression of the IV tube 220 (e.g., by ensuring that the first arm segment 325a and the second arm segment 325b do not add additional resistance to the IV tube 220).

In a third sequence 350, as the first pumping element 310 returns to it starting position (e.g., moves away from the second pumping element 315) the IV tube 220 begins to return to its original (uncompressed) state, which occurs quickly using the pumping segment recovery device 320 as discussed below. Similarly, the pumping segment recovery device 320 also returns to its initial gap width (wᵣₑₛₜ). In some implementations, the pumping segment recovery device 320 has a predetermined elasticity such that the first arm segment 325a and the second arm segment 325b return the gap width (wᵣₑₛₜ) after the first pumping element 310 withdraws from the tube receiving space 329 and no longer contacts the IV tube 220. As the pumping segment recovery device 320 returns to the initial gap width (wᵣₑₛₜ) from the separation width of wₑₓₚ, the pumping segment recovery device 320 applies a force on the IV tube 220 and causes the IV tube 220 to rebound quickly. In particular, after the first pumping element 310 contacts the IV tube 220 received in the tube receiving space 329 and while the first pumping element 310 withdraws from the tube receiving space 329, the first arm segment 325a and the second arm segment 325b contact the IV tube 220 and cause it to return to its original state quickly (e.g., without stiction holding the IV tube 220 together).

In the depicted example, the pumping segment recovery device 320 is configured to be coupled with the first pumping element 310 or the second pumping element 315 of the pumping device. In some implementations, the pumping segment recovery device 320 is coupled opposite the other pumping element. For example, as shown in FIG. 3, the pumping segment recovery device 320 is coupled with the second pumping element 315, which is opposite to the first pumping element 310. In some implementations, the pumping segment recovery device 320 is coupled to a first surface (adjacent to the first pumping element 310) of the second pumping element 315 via a fastener coupling the connecting segment 327 with the first surface of the second pumping element 315. In some implementations, the fastener is a mechanical fastener such as a bolt, a screw, a clamp, etc. Alternatively or additionally, in some implementations, the pumping segment recovery device 320 is coupled to the first surface of the second pumping element 315 via an adhesive and/or weld. Alternatively, in some implementations, the pumping segment recovery device 320 is configured to receive the second pumping element 315 via the tube receiving space 329 between the first and second arm segments 325a and 325b (e.g., before the IV tube 220 is received). In other words, the second pumping element 315 is placed within the tube receiving space 329 of the pumping segment recovery device 320 such that the connecting segment 327 is adjacent to a second surface of the second pumping element 315 opposite to the first surface (which is adjacent to the first pumping element 310). In some implementations, when the second pumping element 315 is within the tube receiving portion 329, the first and second arm segments 325a and 325b apply a pressure and/or clamp on the second pumping element 315 such that it does not move during use. Alternatively or in addition, in some implementations, when the second pumping element 315 is within the tube receiving portion 329, a fastener, adhesive, or weld is used to couple the connecting segment 327 of the pumping segment recovery device 320 to the second surface of the second pumping element 315 and/or couple a portion of the first and second arm segments 325a and 325b to side portions (adjacent sides to the first and second surfaces) of the second pumping element 315.

Turning now to FIG. 4, the pumping segment recovery device 320 is incorporated in an infusion deice, in accordance with some implementations. In FIG. 4, an infusion pump 22 having a body 27 is shown in perspective view, in accordance with various aspects of the present disclosure. The infusion pump 22 is shown with the front door 50 open, showing the upstream fluid line 30 and downstream fluid line 31 in operative engagement with the pump 22. The infusion pump 22 directly acts on a tube 66 (analogous to IV tube 220; FIG. 3) that connects the upstream fluid line 30 to the downstream fluid line 31 to form a continuous fluid conduit, extending from the respective fluid supply 38 (FIG. 1) to the patient 48, through which fluid is acted upon by the pump to move fluid downstream to the patient. Specifically, a pumping mechanism 70 acts as the flow control device of the pump to move fluid though the conduit. The upstream and downstream fluid lines and/or tube 66 may be coupled to a pump cassette or cartridge that is configured to be coupled to the pump 22.

The type of pumping mechanism may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

In some implementations, one or more pumping segment recovery devices 320 are coupled to one or more pumping element of the pumping mechanism and configured to receive the tube 66 (e.g., via respective tube receiving spaces 329; FIG. 3). In some implementations, one or more pumping segment recovery devices 320 are part of, or coupled to door 50. For example, as shown in FIG. 4, a pumping segment recovery device 320 can be coupled to a pump element (e.g., planar surface 91) on the door 50. In some implementations, the one or more pumping segment recovery devices 320 can be coupled with any of the pumping fingers and valve fingers 72, 74, 76, and 78. For example, a pumping segment recovery device 320 can be coupled to each pumping fingers and valve fingers 72, 74, 76, and 78. The pumping segment recovery device 320 is configured to receive the tube 66 when the door 50 of the pumping device is closed. As described above in reference to FIG. 3, the one or more pumping segment recovery devices 320 are configured to rebound tube 66 after it has been compressed by the pumping device.

FIG. 4 further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 1) and the flow control device, so that the connection of the correct fluid supply with the correct pump may be verified before any fluid is pumped to the patient.

With reference still to FIG. 4, an upstream pressure sensor 80 may also be included in the pump 22. The upstream pressure sensor is assigned to the flow control device or pumping mechanism 70 and, in this implementation, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 1) and the flow control device, so that the connection of the correct fluid supply with the correct pump may be verified before any fluid is pumped to the patient.

The pump 22 or a portion of the pump 22 may also be equipped with an electronic data tag or data transmitter. For example, as shown in FIG. 4, pump 22 may be equipped with a data tag 89 or a reader device 90 for providing or receiving infusion data. The data reader devices may include RFID readers (or receivers) or other wireless devices that are compatible with the data tags associated with the fluid containers. A data transmitter may transmit interrogation signals to the electronic data tags 81, 83, 85, 87 associated with the fluid containers for obtaining infusion data from those tags. Although referred to as data transmitting devices or RFID tags or RFID transponders, data transmitting devices may also receive or read data and may also be writable.

Typically, medical tubing (e.g., tube 66) is a disposable product that is used once and then discarded. The medical tubing may be formed from any suitable material, e.g., soft PVC, silicone, thermoplastic vulcanizate (TPV) (ethylene propylene diene monomer (EPDM) +polypropylene (PP)), thermoplastic polyurethane (TPU), thermoplastic styrenic elastomer (TPS) (styrene-butadiene-styrene (SBS)/tyrene-ethylene-butylene-styrene (SEBS)/tyrene-isoprene-rubber (SIS)/tyrene-ethylene-propylene-styrene (SEPS)) and its blending with polyolefin, thermoplastic polyester elastomer (TPEE) (polyether ester) rubber). As shown in FIG. 4, medical tubing 66 may be inserted into or otherwise engaged by pump 22. Pump 22 may include any of Large Volume, patient-controlled analgesia (PCA), ambulatory pump or insulin pump that drive tubing segment(s) to deliver medication or nutrients into a patient's body in controlled amounts. The medical tubing 66 is compressed when the pump door 50 is closed. With the pump door 50 closed, the medical tubing 66 is constrained within a gap 54 and directly contacted by the upstream force sensor 80. Similarly, the medical tube 66 is received within the tube receiving space 329 of each pumping segment recovery device 320. As discussed above, there are many sources of variation in measuring the force on the medical tubing 66 by the sensor 80.

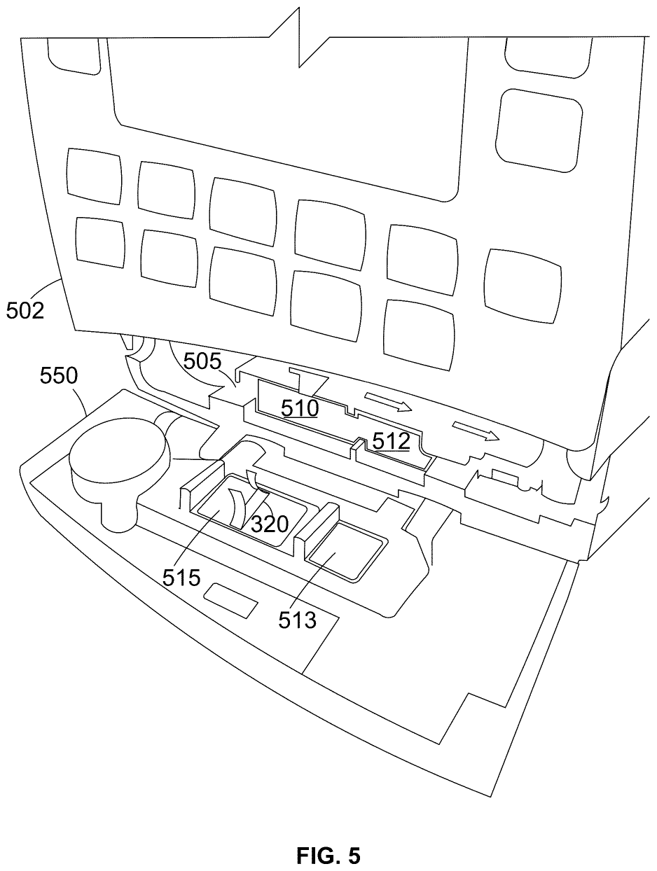

FIG. 5 illustrates an example pumping segment recovery device incorporated in another example pumping device, in accordance with some implementations. According to various implementations, the depicted pumping device 502 is analogous to infusion pumps 22, 24, 26, and 28 described above in reference to FIGS. 1 and 4. In some implementations, the pumping device 502 includes a door 550. The door 550, when opened, grants a user access to one or more pumping elements 510, 512, 513, and 515 of the pumping device 502 and/or an IV tube 220 (FIG. 3). Similarly, the user is able to attach the IV tube 220 to the pumping device 502 (e.g., place the IV tube within the pumping device 502 and/or within a tube receiving portion 329 of a pumping segment recovery device 320 as described above in reference to FIG. 3. In some implementations, the IV tube 66 is received via a tube receiving portion 505 of the pumping device 502. In some implementations, the IV tube 66 is manually placed withing the tube receiving portion 329 of the pumping segment recovery device 320 before the door 550 is closed. Alternatively, in some implementations, the pumping segment recovery device 320 is configured to automatically push the IV tube 66 within the tube receiving portion 329 when the door 550 is closed.

As described above in reference to FIGS. 3 and 4, the pumping segment recovery device 320 can be coupled to one or more pumping elements 510, 512, 513, and 515. For example, the pumping segment recovery device 320 is coupled to a second pumping element 515 of the pumping device 502. In some implementations, the second pumping element 515 is an actuated element (e.g., moves towards and/or away from a first pumping element 510). As described above in reference to FIG. 3, the pumping segment recovery device 320 is configured to expand when a corresponding pumping element 510, 512, 513, or 515 is actuated toward the IV tube 66 (compressing the IV tube) and rebound after the corresponding pumping element 510, 512, 513, or 515. For example, when either the first pumping element 510, the second pumping element 515, or both are actuated, the pumping segment recovery device 320 is expanded to at least a width of a pumping element of a 510, 512, 513, or 515, and after the first pumping element 510, the second pumping element 515, or both withdraw, the pumping segment recovery device 320 rebounds to its initial gap width. When the pumping segment recovery device 320 rebounds, it provides pressure to a portion of the IV tube 66 which cause the IV tube 66 to quickly rebound.

In some implementations, the pumping segment recovery device 320 can be removed from the pumping device 502 when it nears its end of life. This allows for the pumping segment recovery device 320 to be replaced with a new pumping segment recovery device 320. In some implementations, the door 50 can be removed and replaced with another door that includes the pumping segment recovery device 320. This allows for existing pumping devices 502 to be retrofitted and/or reduces the downtime of a pumping device 502 by reducing the downtime required for maintenance.

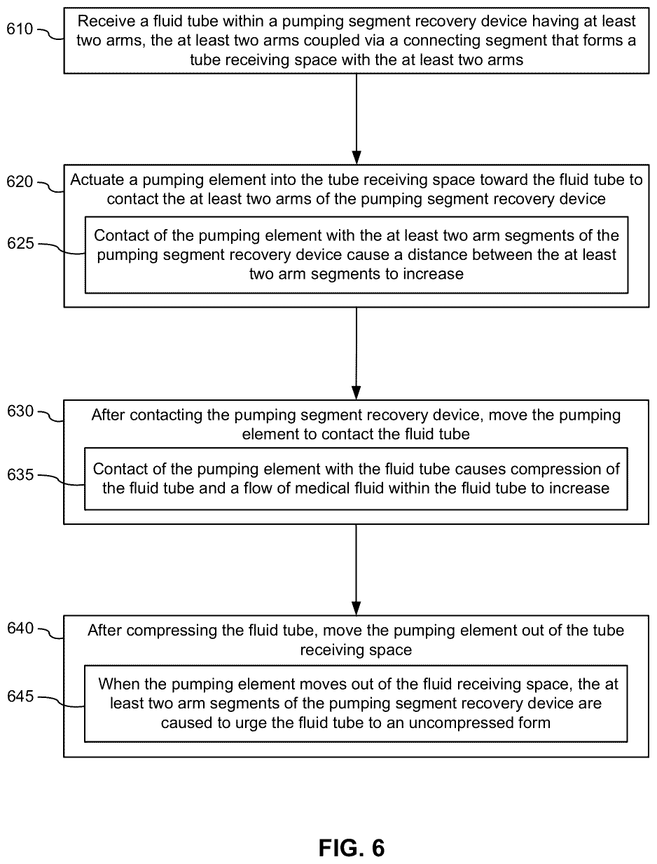

FIG. 6 illustrates a flow diagram of a method for operating a pump including a pumping segment recovery device, in accordance with various implementations. Method 600 can be performed at a medical device described above in reference to FIGS. 1 and 3-5. In particular, the method can be performed at a medical device including a body, a pump mechanism, and a pumping segment recovery device 320. The body is configured to receive and secure an IV tube 66 in an extended position. The IV tube 66 is configured to allow for the transportation of a fluid. As described above in reference to FIGS. 3-5, in some implementations, the pump mechanism includes one or more pumping elements and one or more pumping segment recovery devices. At least some of the operations shown in FIG. 6 correspond to instructions stored in a computer memory or computer-readable storage medium (e.g., storage, ram, and/or memory 61; FIG. 1). Operations 610-650 can also be performed in part using one or more processors and/or using instructions stored in memory or computer-readable medium of an electronic device communicatively coupled to the medical device (e.g., a server or patient care system 20 (FIG. 1) can perform operations 610-650 alone or in conjunction with the one or more processors of the medical device).

The method 600 includes receiving (610) a fluid tube (e.g., IV tube 220; FIG. 3) within a pumping segment recovery device 320 having at least two arms (e.g., a first arm segment 325a and a second arm segment 325b; FIG. 3). The at least two arms are coupled via a connecting segment 327 that forms a tube receiving space 329 with the at least two arms as shown above in FIG. 3. The method 600 further includes actuating (620) a pumping element into the tube receiving space toward the fluid tube to contact the at least two arms of the pumping segment recovery device. Contact of the pumping element with the at least two arm segments of the pumping segment recovery device cause (625) a distance between the at least two arm segments to increase. For example, as shown in the second sequence 330 of FIG. 3, the first pumping element 310 is actuated towards the second pumping element 315, which causes the first and second arm segments 325a and 325b of the pumping segment recovery device 320 to separate by a width of the first pumping element 310.

The method 600 further includes, after contacting the pumping segment recovery device, moving (630) the pumping element to contact the fluid tube. Contact of the pumping element with the fluid tube causes (635) compression of the fluid tube and a flow of medical fluid within the fluid tube to increase. After compressing the fluid tube, the method 600 includes moving (640) the pumping element out of the tube receiving space, such that when the pumping element moves out of the fluid receiving space, the at least two arm segments of the pumping segment recovery device are caused (645) to urge the fluid tube to an uncompressed form. For example, as shown in the third sequence 350 of FIG. 3, the first pumping element 310 is withdrawn from the tube receiving space 329 and returns to its original position, and the first and second arm segments 325a and 325b of the pumping segment recovery device 320 push against opposite side of the IV tube 220 causing the IV tube 220 rebound to its original uncompressed state. Additional examples of the pumping segment recovery device 320 are provided above in reference to FIGS. 3-5.

Many of the above-described example steps of method 700, and related features and applications, may also be controlled by software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

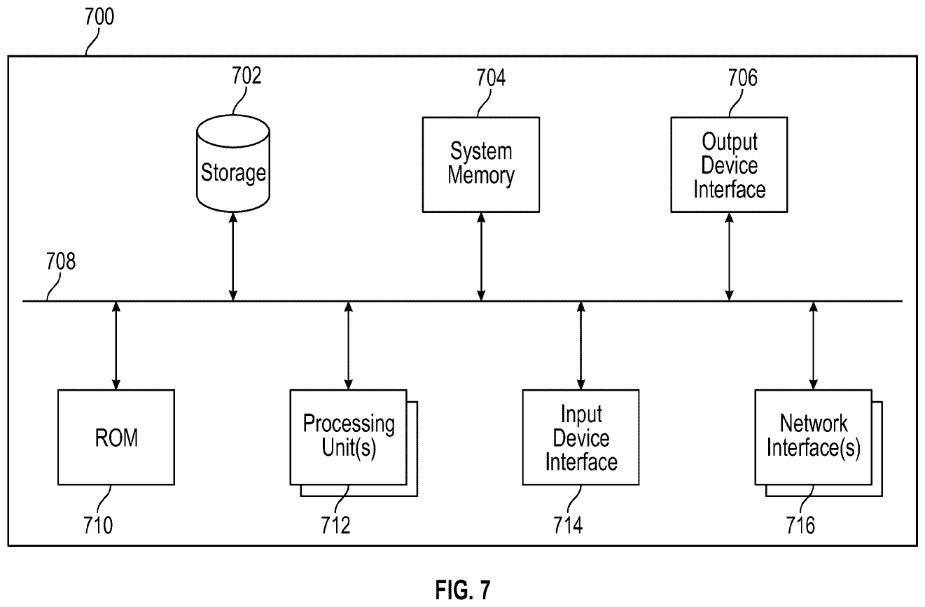

FIG. 7 is a conceptual diagram illustrating an example electronic system 700 for controlling a pump including a pumping segment recovery device, according to aspects of the subject technology. Electronic system 700 may be a specifically configured computing device for execution of software associated with one or more portions or steps of process 700, or components and processes provided by FIGS. 1 through 6, including but not limited to controller 60 of patient care system 20 and/or infusion pumps 22, 24, 26, and 28. Electronic system 700 may be representative, in combination with the disclosure regarding FIGS. 1 through 6.

Electronic system 700 may include various types of computer readable media and interfaces for various other types of computer readable media. In the depicted example, electronic system 700 includes a bus 708, processing unit(s) 712, a system memory 704, a read-only memory (ROM) 710, a permanent storage device 702, an input device interface 714, an output device interface 706, and one or more network interfaces 716. In some implementations, electronic system 700 may include or be integrated with other computing devices or circuitry for operation of the various components and processes previously described.

Bus 708 collectively represents all system, peripheral, and chipset buses that communicatively connect the numerous internal devices of electronic system 700. For instance, bus 708 communicatively connects processing unit(s) 712 with ROM 710, system memory 704, and permanent storage device 702.

From these various memory units, processing unit(s) 712 retrieves instructions to execute and data to process, in order to execute the processes of the subject disclosure. The processing unit(s) can be a single processor or a multi-core processor in different implementations.

ROM 710 stores static data and instructions that are needed by processing unit(s) 712 and other modules of the electronic system. Permanent storage device 702, on the other hand, is a read-and-write memory device. This device is a non-volatile memory unit that stores instructions and data even when electronic system 700 is off. Some implementations of the subject disclosure use a mass-storage device (such as a magnetic or optical disk and its corresponding disk drive) as permanent storage device 702.

Other implementations use a removable storage device (such as a floppy disk, flash drive, and its corresponding disk drive) as permanent storage device 702. Like permanent storage device 702, system memory 704 is a read-and-write memory device. However, unlike storage device 702, system memory 704 is a volatile read-and-write memory, such as a random access memory. System memory 704 stores some of the instructions and data that the processor needs at runtime. In some implementations, the processes of the subject disclosure are stored in system memory 704, permanent storage device 702, and/or ROM 710. From these various memory units, processing unit(s) 712 retrieves instructions to execute and data to process in order to execute the processes of some implementations. Such storage and/or memory devices 702, 704 may be representative of memory 61 of controller 60.

Bus 708 also connects to input and output device interfaces 714 and 706. Input device interface 714 enables the user to communicate information and select commands to the electronic system. Input devices used with input device interface 714 include, e.g., alphanumeric keyboards and pointing devices (also called “cursor control devices”), such as those shown in controller 60 of FIG. 1. Output device interfaces 706 (e.g., shown as a display in the controller 60 of FIG. 1) enables, e.g., the display of images generated by the electronic system 700. Output devices used with output device interface 706 include, e.g., printers and display devices, such as cathode ray tubes (CRT) or liquid crystal displays (LCD). Some implementations include devices such as a touchscreen that functions as both input and output devices.

Also, as shown in FIG. 7, bus 708 also couples electronic system 700 to a network (not shown) through network interfaces 716. Network interfaces 716 may include, e.g., a wireless access point (e.g., Bluetooth or WiFi) or radio circuitry for connecting to a wireless access point. Network interfaces 716 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 700 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user, e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A pumping segment recovery device including a first arm segment, a second arm segment, a tube receiving space between the first arm and the second arm, and a connecting segment coupling the first arm segment with the second arm segment. The connecting segment is arranged below the tube receiving space. The first arm segment includes a first flared end to contact a pumping element before the pumping element contacts a fluid tube received in the tube receiving space, the second arm segment includes a second flared end to contact the pumping element before the pumping element contacts the fluid tube received in the tube receiving space.
Clause 2. The pumping segment recovery device of Clause 1, the first arm segment and the second arm segment are separated by a gap width greater than an outer diameter of the fluid tube received in the tube receiving space. In some implementations, the gap width is equal to or less than a width of the pumping element. In some implementations, the connecting segment separates the first arm and the second arm by a predetermined distance (i.e., the gap width). The predetermined distance is greater than an outer diameter of the fluid tube.
Clause 3. The pumping segment recovery device of Clause 2, the first arm segment and the second arm segment are separate by at least the width of the pumping element when the pumping element engages and contacts the fluid tube received in the tube receiving space.
Clause 4. The pumping segment recovery device of Clause 3, after the pumping element contacts the fluid tube received in the tube receiving space and while the pumping element withdraws from the tube receiving space, the first arm segment and the second arm segment contact the fluid tube received in the tube receiving space.
Clause 5. The pumping segment recovery device of any one of Clause 3 and Clause 4, the pumping segment recovery device has a predetermined elasticity such that the first arm segment and the second arm segment are separated by the gap width after the pumping element withdraws from the tube receiving space and no longer contacts the fluid tube received in the tube receiving space. In other words, the first arm segment and the second arm segment return to the gap width when at a resting state.
Clause 6. The pumping segment recovery device of any one of Clause 1 through Clause 5, the pumping element is a first pumping element, and the pumping segment recovery device is coupled to a second pumping element opposite the first pumping element.
Clause 7. The pumping segment recovery device of Clause 6, the pumping segment recovery device is coupled to a first surface of the second pumping element via a fastener coupling the connecting segment with the first surface of the second pumping element, the first surface of the second pumping element adjacent to the first pumping element.
Clause 8. The pumping segment recovery device of Clause 6, the second pumping element is received via the tube receiving space between the first arm segment and the second arm segment before the fluid tube received in the tube receiving space, and the first arm segment and the second arm segment couple the second pumping element to the pumping segment recovery device.
Clause 9. The pumping segment recovery device of any one of Clause 6 through Clause 8, the second pumping element is part of an infusion device.
Clause 10. The pumping segment recovery device of Clause 9, the part of the infusion device is a door.
Clause 11. The pumping segment recovery device of Clause 9, the part of the infusion device is portion of a pump mechanism of the infusion device.
Clause 12. The pumping segment recovery device of any one of Clause 1 through Clause 11, the pumping segment recovery device is formed of a metallic material.
Clause 13. The pumping segment recovery device of any one of Clause 1 through Clause 12, a distance between the first flared end and the second flared end is equal to or greater than a width of the pumping element.
Clause 14. The pumping segment recovery device of any one of Clause 1 through Clause 13, the first arm segment has a first s-shape and the second arm segment has a second s-shape opposite the first s-shape.
Clause 15. An infusion device includes a housing, a fluid path to direct a fluid from a container via a fluid tube, and a pumping segment recovery device mounted along the fluid path within the housing. The pumping segment recovery device includes a first arm segment, a second arm segment, a tube receiving space between the first arm segment and the second arm segment, and a connecting segment coupling the first arm segment with the second arm segment. The connecting segment is arranged below the tube receiving space. The first arm segment includes a first flared end to contact a pumping element before the pumping element contacts the fluid tube received in the tube receiving space, and the second arm segment includes a second flared end to contact the pumping element before the pumping element contacts the fluid tube received in the tube receiving space.
Clause 16. The infusion device of Clause 15, the pumping segment recovery device is configured in accordance with any one of Clause 2 through Clause 14.
Clause 17. A method of compressing a fluid tube includes receiving a fluid tube within a pumping segment recovery device having at least two arm segments. The at least two arm segments are coupled via a connecting segment that forms a tube receiving space with the at least two arm segments. The method also includes actuating a pumping element into the tube receiving space toward the fluid tube to contact the at least two arm segments of the pumping segment recovery device. The contact of the pumping element with the at least two arm segments causes a distance between the at least two arms to increase. The method includes after the pumping element contacts the pumping segment recovery device, moving the pumping element to contact the fluid tube. The contact of the pumping element with the fluid tube causes compression of the fluid tube. The method further includes, after the pumping element compresses the fluid tube, moving the pumping element out of the tube receiving space. When the pumping element moves out of the fluid receiving space, the at least two arm segments of the pumping segment recovery device are caused to urge the fluid tube to an uncompressed form.
Clause 18. The method of Clause 17, the pumping segment recovery device is configured in accordance with any one of Clause 1 through Clause 14.

### Further Consideration

In some implementations, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

1. A pumping segment recovery device, comprising:
a first arm segment;
a second arm segment;
a tube receiving space between the first arm and the second arm; and
a connecting segment coupling the first arm segment with the second arm segment, the connecting segment arranged below the tube receiving space,
wherein the first arm segment includes a first flared end to contact a pumping element before the pumping element contacts a fluid tube received in the tube receiving space,
wherein the second arm segment includes a second flared end to contact the pumping element before the pumping element contacts the fluid tube received in the tube receiving space.

2. The pumping segment recovery device of claim 1, wherein the first arm segment and the second arm segment are separated by a gap width greater than an outer diameter of the fluid tube received in the tube receiving space.

3. The pumping segment recovery device of claim 2, wherein the first arm segment and the second arm segment separate by at least the width of the pumping element when the pumping element engages and contacts the fluid tube received in the tube receiving space.

4. The pumping segment recovery device of claim 3, wherein after the pumping element contacts the fluid tube received in the tube receiving space and while the pumping element withdraws from the tube receiving space, the first arm segment and the second arm segment contact the fluid tube received in the tube receiving space.

5. The pumping segment recovery device of claim 3, wherein the pumping segment recovery device has a predetermined elasticity such that the first arm segment and the second arm segment are separated by the gap width after the pumping element withdraws from the tube receiving space and no longer contacts the fluid tube received in the tube receiving space.

6. The pumping segment recovery device of claim 1, wherein:
the pumping element is a first pumping element; and
the pumping segment recovery device is coupled to a second pumping element opposite the first pumping element.

7. The pumping segment recovery device of claim 6, wherein the pumping segment recovery device is coupled to a first surface of the second pumping element via a fastener coupling the connecting segment with the first surface of the second pumping element, the first surface of the second pumping element adjacent to the first pumping element.

8. The pumping segment recovery device of claim 6, wherein:
the second pumping element is received via the tube receiving space between the first arm segment and the second arm segment before the fluid tube received in the tube receiving space; and
the first arm segment and the second arm segment couple the second pumping element to the pumping segment recovery device.

9. The pumping segment recovery device of claim 6, wherein the second pumping element is part of an infusion device.

10. The pumping segment recovery device of claim 9, wherein the part of the infusion device is a door.

11. The pumping segment recovery device of claim 9, wherein the part of the infusion device is portion of a pump mechanism of the infusion device.

12. The pumping segment recovery device of claim 1, wherein the pumping segment recovery device is formed of a metallic material.

13. The pumping segment recovery device of claim 1, wherein a distance between the first flared end and the second flared end is equal to or greater than a width of the pumping element.

14. The pumping segment recovery device of claim ,1 wherein the first arm segment has a first s-shape and the second arm segment has a second s-shape opposite the first s-shape.

15. An infusion device comprising:
a housing;
a fluid path to direct a fluid from a container via a fluid tube;
a pumping segment recovery device mounted along the fluid path within the housing, the pumping segment recovery device comprising:
a first arm segment;
a second arm segment;
a tube receiving space between the first arm segment and the second arm segment; and
a connecting segment coupling the first arm segment with the second arm segment, the connecting segment arranged below the tube receiving space,
wherein the first arm segment includes a first flared end to contact a pumping element before the pumping element contacts the fluid tube received in the tube receiving space,
wherein the second arm segment includes a second flared end to contact the pumping element before the pumping element contacts the fluid tube received in the tube receiving space.

16. A method of compressing a fluid tube comprising:
receiving a fluid tube within a pumping segment recovery device having at least two arm segments, wherein the at least two arm segments are coupled via a connecting segment that forms a tube receiving space with the at least two arm segments;
actuating a pumping element into the tube receiving space toward the fluid tube to contact the at least two arm segments of the pumping segment recovery device, wherein contact of the pumping element with the at least two arm segments of the pumping segment recovery device cause a distance between the at least two arm segments to increase;
after contacting the pumping segment recovery device, moving the pumping element to contact the fluid tube, wherein contact of the pumping element with the fluid tube causes compression of the fluid tube; and
after compressing the fluid tube, moving the pumping element out of the tube receiving space, wherein, when the pumping element moves out of the fluid receiving space, the at least two arm segments of the pumping segment recovery device are caused to urge the fluid tube to an uncompressed form.

17. The method of claim 16, wherein the first arm segment and the second arm segment are separated by a gap width greater than an outer diameter of the fluid tube received in the tube receiving space.

18. The method of claim 16, wherein:
the pumping element is a first pumping element; and
the pumping segment recovery device is coupled to a second pumping element opposite the first pumping element.

19. The infusion device of claim 15, wherein the first arm segment and the second arm segment are separated by a gap width greater than an outer diameter of the fluid tube received in the tube receiving space.

20. The infusion device of claim 15, wherein:
the pumping element is a first pumping element; and
the pumping segment recovery device is coupled to a second pumping element opposite the first pumping element.
